(19) United States
(12) Patent Application Publication
(10) Pub. No.: US 2026/0248560 A1
(43) Pub. Date: Aug. 27, 2026

(54) APPARATUS AND METHOD FOR TRACKING A DEVICE HAVING A MAGNET

(71) Applicants: Singapore University of Technology and Design, Singapore (SG); Singapore Health Services Pte Ltd, Singapore (SG)

(72) Inventors: Shaohui FOONG, Singapore (SG); Thi Ngoc NGUYEN, Singapore (SG); Qianfeng JIANG, Singapore (SG); Muhammad Syafiq LUTFI, Singapore (SG); Yi Shu Billy WONG, Singapore (SG); Foong Sin Alice CHUA, Singapore (SG); Peijin Esther Monica FAN, Singapore (SG)

(21) Appl. No.: 18/992,132

(22) PCT Filed: Jul. 7, 2023
(86) PCT No.: PCT/SG2023/050479

§ 371 Date: Jan. 7, 2025

(30) Foreign Application Priority Data
Jul. 8, 2022 (SG) 10202250419N

(51) Int. Cl. A61B 34/20; A61B 5/06

(52) U.S. Cl. CPC A61B 34/20; A61B 5/062; A61B 2034/2051

(57) ABSTRACT

The present disclosure generally relates to an apparatus and method for tracking a device having a magnet. The apparatus comprises magnetic sensors arranged longitudinally and configured for measuring a magnetic field from the magnet as the device moves along a trajectory. A processor receives the magnetic field measurements of the magnet; determines, for each magnetic sensor, axial magnetic field components directed along mutually orthogonal axial directions; selects the axial magnetic field components that satisfy a threshold predefined for each axial direction; calculates a pose of the magnet using the selected axial magnetic field components and predetermined magnetic moments for the magnetic sensors; and tracks the device using the calculated poses of the magnet as the device moves along the trajectory.

### CROSS REFERENCE TO RELATED APPLICATIONS

The present disclosure claims the benefit of Singapore patent application Ser. No. 10/202,250419N filed on 8 Jul. 2022, which is incorporated in its entirety by reference herein.

### TECHNICAL FIELD

The present disclosure generally relates to an apparatus and method for tracking a device having a magnet. More particularly, the present disclosure describes various embodiments of an apparatus and a method for tracking a medical instrument having a magnet, such as a catheter with an embedded magnet.

### BACKGROUND

Medical devices are often inserted into the human body in various medical diagnosis and treatment procedures, such as catheters like a nasogastric tube or ventriculostomy catheter. For example, ventriculostomy and nasogastric tube insertion procedures are done prior to commencing nutrients or medication administration. However, such procedures are often performed “blind” without visual aids, making it difficult to confirm whether the instrument tip is correctly positioned in the right place. Incorrect positioning of the device may lead to grave consequences. Indeed, 21 deaths and 79 other cases of harm due to misplaced nasogastric tubes into the respiratory tract were reported in the United Kingdom between September 2005 and 31 Mar. 2010. Thus, real-time tracking of the device moving through the human body and confirming the tip position is desirable in medical diagnosis and treatments.

Existing methods to verify the location of the instrument tip inside the human body after insertion include performing a pH test of the nasogastric tube aspirate and/or a chest X-ray. However, these methods only detect and identify the abnormal occurrences after the tube has been inserted. A delay to nutrition feeds or medication administration and a repeat of the tube insertion are to be expected.

With the proliferation of low-cost magnetic sensors, tracking methods based on magnets have emerged as effective ways to track the catheter tip in nasogastric intubation and ventriculostomy procedures in real time without requiring any power for the magnet. However, the tracking range of magnetic sensors is constrained by the sensor sensitivity, with low-cost magnetic sensors having a maximum tracking range of 150 mm to 250 mm. This tracking range can only cover the average length from the cervical oesophagus to the oesophagogastric junction if the nasogastric tube is inserted correctly. To avoid deep lung misplacement, detection of the tube tip must pass this oesophagogastric junction, which is beyond the maximum tracking range of current magnet-based methods.

Therefore, in order to address or alleviate at least one of the aforementioned problems and/or disadvantages, there is a need to provide an improved apparatus and method for tracking a device having a magnet.

### SUMMARY

According to a first aspect of the present disclosure, there is an apparatus for tracking a device having a magnet. The apparatus comprises: a processor; and a plurality of magnetic sensors arranged along a longitudinal direction based on a trajectory of the device, the magnetic sensors configured for measuring a magnetic field from the magnet as the device moves along the trajectory. The processor is configured for: receiving, from the magnetic sensors, the magnetic field measurements of the magnet; determining, for each magnetic sensor, axial magnetic field components from the magnetic field measurements from the respective magnetic sensor, the axial magnetic field components directed along mutually orthogonal axial directions of the magnetic sensor; selecting, for each magnetic sensor, the axial magnetic field components that satisfy a magnetic field threshold predefined for each axial direction of each magnetic sensor; calculating a pose of the magnet using the selected axial magnetic field components and predetermined magnetic moments for the magnetic sensors; and tracking the device using the calculated poses of the magnet as the device moves along the trajectory.

According to a second aspect of the present disclosure, there is a method for tracking a device having a magnet. The method comprises: arranging a plurality of magnetic sensors along a longitudinal direction based on a trajectory of the device; controlling movement of the device along the trajectory; measuring, by the magnetic sensors, a magnetic field from the magnet as the device moves along the trajectory; receiving, by a processor and from the magnetic sensors, the magnetic field measurements of the magnet; determining, by the processor and for each magnetic sensor, axial magnetic field components from the magnetic field measurements from the respective magnetic sensor, the axial magnetic field components directed along mutually orthogonal axial directions of the magnetic sensor; selecting, by the processor and for each magnetic sensor, the axial magnetic field components that satisfy a magnetic field threshold predefined for each axial direction of each magnetic sensor; calculating, by the processor, a pose of the magnet using the selected axial magnetic field components and predetermined magnetic moments for the magnetic sensors; and tracking, by the processor, the device using the calculated poses of the magnet as the device moves along the trajectory.

According to a third aspect of the present disclosure, there is a computerized method for tracking a device having a magnet. The computerized method comprises: receiving magnetic field measurements of the magnet as the device moves along the trajectory, the magnetic field measurements measured by a plurality of magnetic sensors arranged along a longitudinal direction based on the trajectory; determining, for each magnetic sensor, axial magnetic field components from the magnetic field measurements from the respective magnetic sensor, the axial magnetic field components directed along mutually orthogonal axial directions of the magnetic sensor; selecting, for each magnetic sensor, the axial magnetic field components that satisfy a magnetic field threshold predefined for each axial direction of each magnetic sensor; calculating a pose of the magnet using the selected axial magnetic field components and predetermined magnetic moments for the magnetic sensors; and tracking the device using the calculated poses of the magnet as the device moves along the trajectory.

An apparatus and method for tracking a device having a magnet according to the present disclosure are thus disclosed herein. Various features and advantages of the present disclosure will become more apparent from the following detailed description of the embodiments of the present disclosure, by way of non-limiting examples only, along with the accompanying drawings.

### BRIEF DESCRIPTION OF THE DRAWINGS

FIGS. 1A and 1B are illustrations of an apparatus for tracking a device having a magnet.

FIG. 2 is an illustration of a magnetic dipole model.

FIGS. 3A and 3B are illustrations calibrating an arrangement of magnetic sensors of the apparatus.

FIGS. 4A to 4D are illustrations of threshold schemes for the magnetic sensors.

FIGS. 5A and 5B are tables showing simulation results from using the threshold schemes.

FIG. 6 is a table showing the magnetic moments for the magnetic sensors.

FIGS. 7A and 7B are illustrations of a numerical simulation for measuring rebars using different frequencies.

FIGS. 7A and 7B are illustrations of results from an experiment using the apparatus.

FIGS. 8A and 8B are further illustrations of results from the experiment using the apparatus.

### DETAILED DESCRIPTION

For purposes of brevity and clarity, descriptions of embodiments of the present disclosure are directed to an apparatus and method for tracking a device having a magnet, in accordance with the drawings. While parts of the present disclosure will be described in conjunction with the embodiments provided herein, it will be understood that they are not intended to limit the present disclosure to these embodiments. On the contrary, the present disclosure is intended to cover alternatives, modifications and equivalents to the embodiments described herein, which are included within the scope of the present disclosure as defined by the appended claims. Furthermore, in the following detailed description, specific details are set forth in order to provide a thorough understanding of the present disclosure. However, it will be recognized by an individual having ordinary skill in the art, i.e. a skilled person, that the present disclosure may be practiced without specific details, and/or with multiple details arising from combinations of features of particular embodiments. In a number of instances, well-known systems, methods, procedures, and components have not been described in detail so as to not unnecessarily obscure features of the embodiments of the present disclosure.

In embodiments of the present disclosure, depiction of a given element or consideration or use of a particular element number in a particular figure or a reference thereto in corresponding descriptive material can encompass the same, an equivalent, or an analogous element or element number identified in another figure or descriptive material associated therewith.

References to “an embodiment/example”, “another embodiment/example”, “some embodiments/examples”, “some other embodiments/examples”, and so on, indicate that the embodiment(s)/example(s) so described may include a particular feature, structure, characteristic, property, element, or limitation, but that not every embodiment/example necessarily includes that particular feature, structure, characteristic, property, element or limitation. Furthermore, repeated use of the phrase “in an embodiment/example” or “in another embodiment/example” does not necessarily refer to the same embodiment/example.

The terms “comprising”, “including”, “having”, and the like do not exclude the presence of other features/elements/steps than those listed in an embodiment. Recitation of certain features/elements/steps in mutually different embodiments does not indicate that a combination of these features/elements/steps cannot be used in an embodiment.

As used herein, the terms “a” and “an” are defined as one or more than one. The use of “/” in a figure or associated text is understood to mean “and/or” unless otherwise indicated. The term “set” is defined as a non-empty finite organization of elements that mathematically exhibits a cardinality of at least one (e.g. a set as defined herein can correspond to a unit, singlet, or single-element set, or a multiple-element set), in accordance with known mathematical definitions. The recitation of a particular numerical value or value range herein is understood to include or be a recitation of an approximate numerical value or value range.

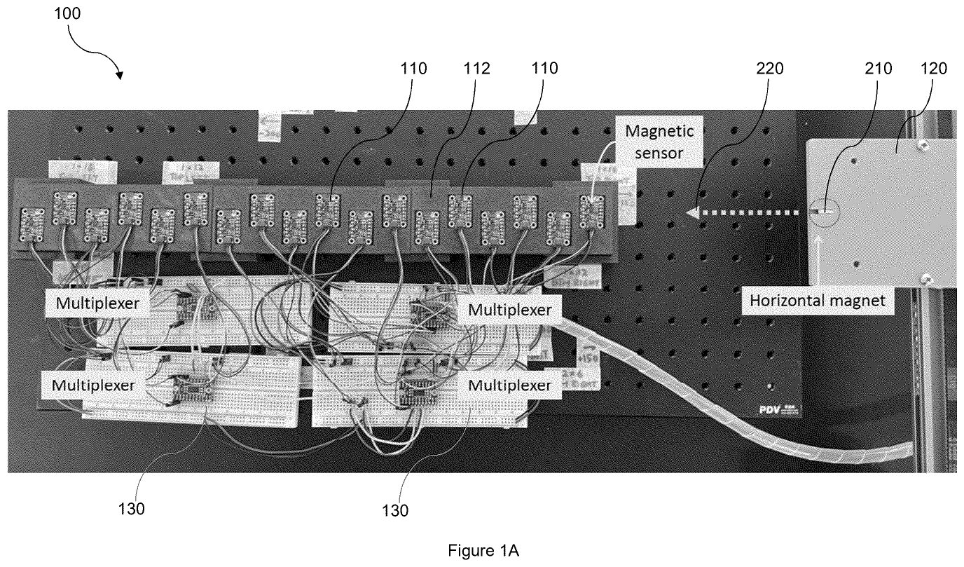

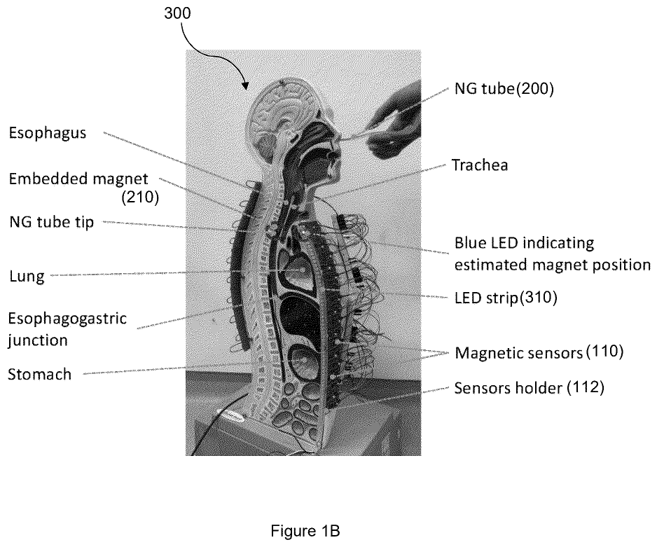

Representative or exemplary embodiments of the present disclosure describe an apparatus 100 for tracking a device 200 having a magnet 210, with reference to FIG. 1A. For example, the magnet 210 is a permanent magnet that may be embedded at the tip of the device 200. In some embodiments, the device 200 is a medical instrument that is inserted into the human body. FIG. 1B shows an anatomical human model 300 with the device 200 being a catheter such as a nasogastric tube. The trajectory 220 of the device 200 includes the insertion path from the cervical oesophagus to the oesophagogastric junction.

The apparatus 100 includes a plurality of magnetic sensors 110 arranged along a longitudinal direction based on a trajectory 220 of the device 200. For example, the longitudinal direction is horizontal and along the x-axis. The magnetic sensors 110 are configured for measuring a magnetic field from the magnet 210 as the device 200 moves along the trajectory 220. The magnet 220 generates the magnetic field and the magnetic flux density of the magnetic field can be detected by the magnetic sensors 110 located outside of the human body.

The apparatus 100 further includes a processor configured for processing the magnetic field measurements of the magnet 210. More specifically, the processor is configured for performing a computer-implemented or computerized method for tracking the device 200 having the magnet 210. The processor executes instructions, codes, computer programs, and/or scripts, and includes suitable logic, circuitry, and/or interfaces to execute such operations or steps. Some non-limiting examples of the processor include an application-specific integrated circuit (ASIC) processor, a reduced instruction set computing (RISC) processor, a complex instruction set computing (CISC) processor, a field-programmable gate array (FPGA), and the like. While instructions may be executed by a processor, the instructions may be executed simultaneously, serially, or otherwise executed by one or multiple processors (e.g. in a multi-core configuration).

Representative or exemplary embodiments of the present disclosure also describe a method for tracking a device 200 having a magnet 210. The method may be performed by the apparatus 100 as shown in FIG. 1 or any other suitable apparatus.

The method includes arranging a plurality of magnetic sensors 110 along a longitudinal direction based on the trajectory 220 of the device 200. The method includes controlling movement of the device 200 along the trajectory 200. For example, the apparatus 100 includes a gantry system 120 for controlling movement of the device 200 along the longitudinal direction (horizontal x-axis) and optionally along the transverse direction (vertical y-axis). The method includes measuring, by the magnetic sensors 110, a magnetic field from the magnet 210 as the device 200 moves along the trajectory 220. The method further includes various steps performed by the processor to process the magnetic field measurements of the magnet 210.

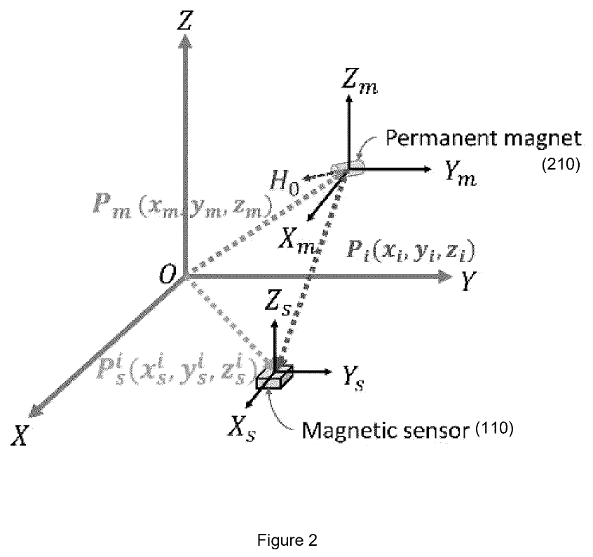

As the distances of the magnetic sensors 110 from the magnet 210 are much larger than the dimensions of the magnet 210, for example a ring magnet with 3.18 mm outer diameter×1.59 mm inner diameter×1.59 mm length, the magnet 210 can be treated as a magnetic dipole. In the magnetic dipole model as shown in FIG. 2, the magnetic source is represented as a single dipole at the geometric centre of the magnet 210 (Pₘ) and the orientation of the dipole is a unit vector H₀ aligned to the magnetization axis of the magnet 210. The measured magnetic field is dependent on the magnetic moment, i.e. the total magnetic dipole moment, the positions of the magnetic sensors 110, and the position and orientation of the magnet 210, as shown in Equation 1.

$${B}_{i} = {M}_{T}^{i} ( \frac{3 ( {H}_{0} \cdot {P}_{i} ) {P}_{i}}{{R}_{i}^{5}} - \frac{{H}_{0}}{{R}_{i}^{3}} ) , i = 1 , 2 , … N \qquad ( Equation 1 )$$

Bᵢ is the magnetic field measured by the i^{th} magnetic sensor 110 when the magnet 210 is at the position Pₘ and has an orientation H₀. M_{T}ⁱ is the magnetic moment for the i^{th} magnetic sensor 110 and can be determined through calibration of the magnetic sensors 110 with the magnet 210. Pᵢ is the vector from Pₘ to the i^{th} magnetic sensor 110 positioned at Pₛⁱ(xₛⁱ, yₛⁱ, zₛⁱ). Rᵢ is the distance between the i^{th} magnetic sensor 110 and the magnet 210. The measured magnetic field Bᵢ and magnetic moment M_{T}ⁱ are used to calculate the pose of the magnet 210, i.e. the magnet position Pₘ and orientation H₀, by solving the non-linear Equation 1.

In some embodiments, the processor is configured for calibrating the magnetic sensors 110 with the magnet 210 to determine the magnetic moment for each magnetic sensor 110. In this calibration process, the actual position Pₘ and orientation H₀ of the magnet 210 are known. The magnetic field measurement by each magnetic sensor 110 is fed into Equation 1 to solve for the unknown magnetic moment for each magnetic sensor 110. More particularly, the magnetic moment is determined for each magnetic sensor 110, unlike current magnet-based methods wherein the total magnetic dipole moment was identified as a single value for all the sensors.

The magnetic moment for each magnetic sensor 110 may be optimized by iteratively adjusting the magnetic moment to minimize the root-mean-square error (RMSE) with respect to a known trajectory 220 of the magnet 210. As shown in Equation 2, the RMSE is calculated between actual positions of the magnet 210 and estimated positions of the magnet 210 determined using the magnetic sensors 110. More specifically, the RMSE is first calculated using the initial magnetic moment obtained from Equation 1. Starting from the initial magnetic moment, the magnetic moment is iteratively adjusted and the RMSE is calculated for each magnetic moment. The magnetic moment with the smallest RMSE is finalized as the magnetic moment for the respective magnetic sensor 110.

$$RMSE = \sqrt{\frac{{\sum }_{i = 1}^{N} {( \vert {P}_{actual} - {P}_{estimated} \vert )}^{2}}{N}} \qquad ( Equation 2 )$$

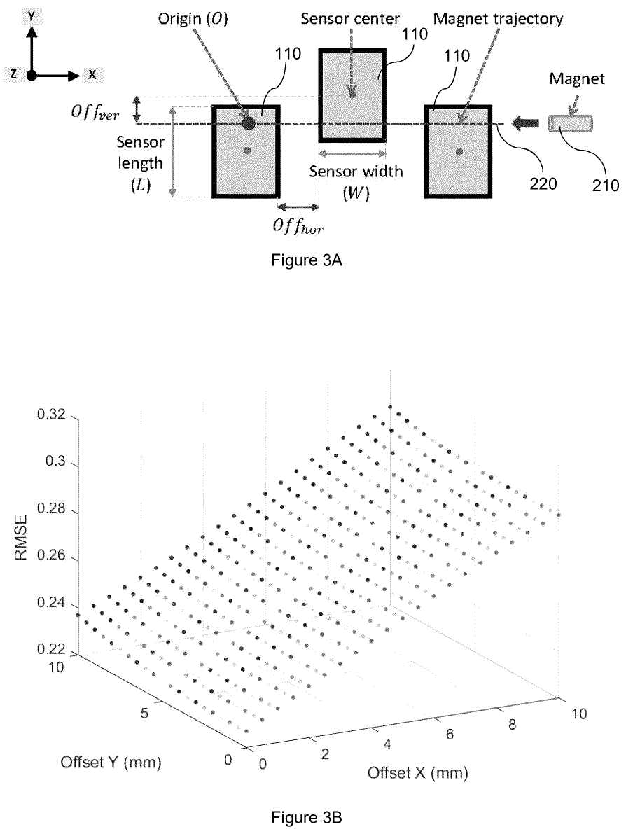

The processor may be configured for calibrating the magnetic sensors 110 with the magnet 210 by adjusting the arrangement of the magnetic sensors 110. For example as shown in FIG. 3A, the spatial arrangement of the magnetic sensors 110 is mapped to a rectangular grid comprising longitudinal and transverse offsets between the magnetic sensors 110 with respect to the origin. The longitudinal direction refers to the horizontal x-axis and the transverse direction refers to the vertical y-axis. The transverse or vertical offset of a magnetic sensor 110 refers to the distance between the centre of the magnetic sensor 110 and the y-axis value of the origin. The longitudinal or horizontal offset between adjacent magnetic sensors 110 refers to the distance along the x-axis between the two nearest sides of the adjacent sensors 110. For example, if two magnetic sensors 110 are in contact with each other, the longitudinal offset is zero.

Adjusting the arrangement of the magnetic sensors 110 includes adjusting the transverse offsets of the magnetic sensors 110 from the longitudinal direction and/or adjusting the longitudinal offsets of the magnetic sensors 110 along the longitudinal direction. The transverse offsets and/or longitudinal offsets may be optimized by iteratively adjusting the arrangement of the magnetic sensors 110 to minimize the RMSE with respect to a known trajectory 220 of the magnet 210. More specifically, the arrangement is iteratively adjusted and the RMSE is calculated for each arrangement using Equation 2. The arrangement with the smallest RMSE is finalized as the one with the optimized transverse offsets and/or longitudinal offsets of the magnetic sensors 110.

In some embodiments as shown in FIG. 1, the magnetic sensors 110 are alternately arranged (or in a zig-zag manner) along the longitudinal direction, each magnetic sensor 110 having a transverse offset from the longitudinal direction. The transverse offsets of the magnetic sensors 110 may be in equal magnitudes along alternate transverse directions. For example, a magnetic sensor 110 may be +1 mm offset along the y-axis and the next magnetic sensor 110 may be −1 mm offset along the y-axis. Adjacent magnetic sensors 110 may have longitudinal offsets from each other in equal magnitudes along the longitudinal direction. For example, the magnitude of the longitudinal offsets is zero, i.e. adjacent magnetic sensors 110 are in contact with each other. The tracking range of the magnetic sensors 110 can be extended or shortened by adding or removing magnetic sensors 110 using the same transverse and longitudinal offsets without incurring additional computational costs.

A numerical simulation was performed to determine the optimal transverse offset in the range [0, 10] mm and the optimal longitudinal offset in the range [0,10] mm, with an increment of each offset value by 0.5 mm. A total of 21×21=441 arrangements of magnetic sensors 110 were generated in the numerical simulation. The RMSE or localization error for each arrangement having a unique pair of transverse and longitudinal offsets was calculated, as shown in FIG. 3B. It was found that the arrangement with a transverse offset of 1 mm and a longitudinal offset of 0 mm resulted in the smallest RMSE of 0.227 mm and the highest localization accuracy.

With the optimized magnetic moments and spatial arrangement of the magnetic sensors 110, the magnetic field measurements of the magnet 210 can be used to track the device 200 with high localization accuracy. As mentioned above, the processor performs various steps to process the magnetic field measurements of the magnet 210.

The processor receives, from the magnetic sensors 110, the magnetic field measurements of the magnet 210. For example, the apparatus 100 includes a set of multiplexers 130 connected between the magnetic sensors 110 and the processor. The multiplexers 130 are configured to connect and interface the magnetic sensors 110 with the processor, such as through the inter-integrated controller (12C) serial communication protocol.

The processor determines, for each magnetic sensor 110, axial magnetic field components from the magnetic field measurements from the respective magnetic sensor 110. The axial magnetic field components are directed along mutually orthogonal axial directions of the magnetic sensor 110. More specifically, the magnetic flux density Bᵢ measured by each i^{th} magnetic sensor 110 has three axial magnetic field components-Bₓⁱ along the x-axis, By along the y-axis, and B_{z}ⁱ along the z-axis. The axial magnetic field components may also be referred to as the axial sensing channels.

Only the useful axial magnetic field components from the magnetic sensors 110 are selected for localizing the magnet 210. This reduces the computational costs for processing the magnetic flux density measured by the magnetic sensors 110, as opposed to the existing method of processing all the magnetic flux density measurements which require more computational resources. Moreover, if the magnetic sensors 110 are arranged according to the optimal transverse offset of 1 mm and optimal longitudinal offset of 0 mm, the resultant array would be highly dense with a large number of magnetic sensors 110 required to track the magnet 210 along the trajectory 220. Processing all the axial magnetic field components from this large number of magnetic sensors 110 would be computationally intensive. Hence, selecting the useful axial magnetic field components reduces the burden placed on the computational resources for processing the magnetic flux density read by all the magnetic sensors 110, thereby providing fine-grained filtering of useful magnetic flux density in magnet localization and reduce the localization time.

Together with the magnetic moment obtained from the magnetic sensor calibration, the magnetic dipole model can be fine-granted constructed based on both the magnetic sensor level and the axial magnetic field components of each magnetic sensor 110, as shown in Equation 3.

$${B}_{a x i a {l}_{k}}^{i} ( {P}_{m} , {H}_{0} ) = {M}_{T}^{i} ( \frac{3 ( {H}_{0} \cdot {P}_{i} ) {P}_{i}}{{R}_{i}^{5}} - \frac{{H}_{0}}{{R}_{i}^{3}} ) , i = 1 , 2 , … N \qquad ( Equation 3 )$$

$${B}_{a x i a {l}_{k}}^{i}$$

is the magnetic field measured by the k^{th} axial magnetic field component of the i^{th} magnetic sensor 110 when the magnet 210 is at the position Pₘ and has an orientation H₀. M_{T}ⁱ is the magnetic moment of the i^{th} magnetic sensor 110 and can be determined through calibration of the magnetic sensors 110 with the magnet 210. Pᵢ is the vector from Pₘ to the i^{th} magnetic sensor 110 positioned at Pₛⁱ (xₛⁱ, yₛⁱ, zₛⁱ). Rᵢ is the distance between the i^{th} magnetic sensor 110 and the magnet 210. The measured axial magnetic field component

$${B}_{a x i a {l}_{k}}^{i}$$

and magnetic moment M_{T}ⁱ are used to calculate the pose of the magnet 210 by solving the non-linear Equation 3.

Using a non-linear optimization algorithm, such as the Levenberg-Marquardt algorithm, a cost function can be defined as shown in Equation 4 and minimized to compute the pose of the magnet 210. More specifically, an arbitrary initial guess of the magnet pose is first fed into the algorithm to compute the next magnet pose, then the computed magnet pose at time t-1 is used as an initial estimation of the magnet pose at the time t.

$$C = {\sum }_{i = 1}^{N} {[ {B}_{k}^{i} ( measured ) - {B}_{k}^{i} ( model ) ]}^{2} \qquad ( Equation 4 )$$

The selection of axial magnetic field components or sensing channels is based on predefined threshold conditions. More specifically, the processor selects, for each magnetic sensor 110, the axial magnetic field components that satisfy a magnetic field threshold predefined for each axial direction of each magnetic sensor 110. For each magnetic sensor 110, the magnetic field threshold predefined for each of the x,y,z-axes axial magnetic field components. If an axial magnetic field component or sensing channel satisfies the corresponding magnetic field threshold, then it is selected and may be referred to as an attentive sensing channel. Otherwise, it is not selected and may be referred to as an inattentive sensing channel.

The attentiveness of an axial magnetic field component of a magnetic sensor 110 depends on the distance between the magnetic sensor 110 and the magnet 210. As such, an axial magnetic field component of a magnetic sensor 110 can become attentive when the magnet 210 is within its axial sensing range and become inattentive when the magnet 210 is out of range. By treating each axial magnetic field component as an independent sensing channel, useful magnetic flux densities can be filtered by selecting the attentive axial magnetic field components for magnet localization. The axial magnetic field components are selected using magnetic field thresholds that fall under a single-threshold scheme or a double-threshold scheme.

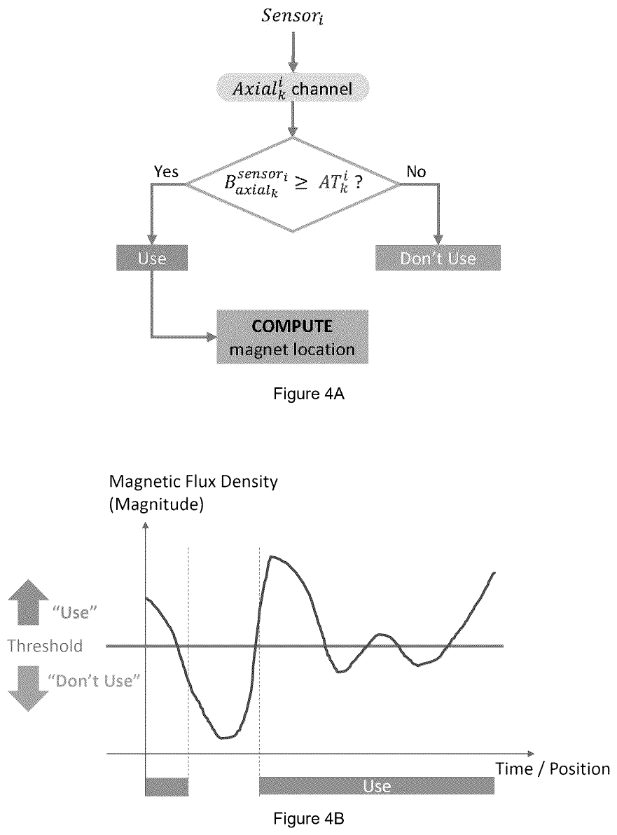

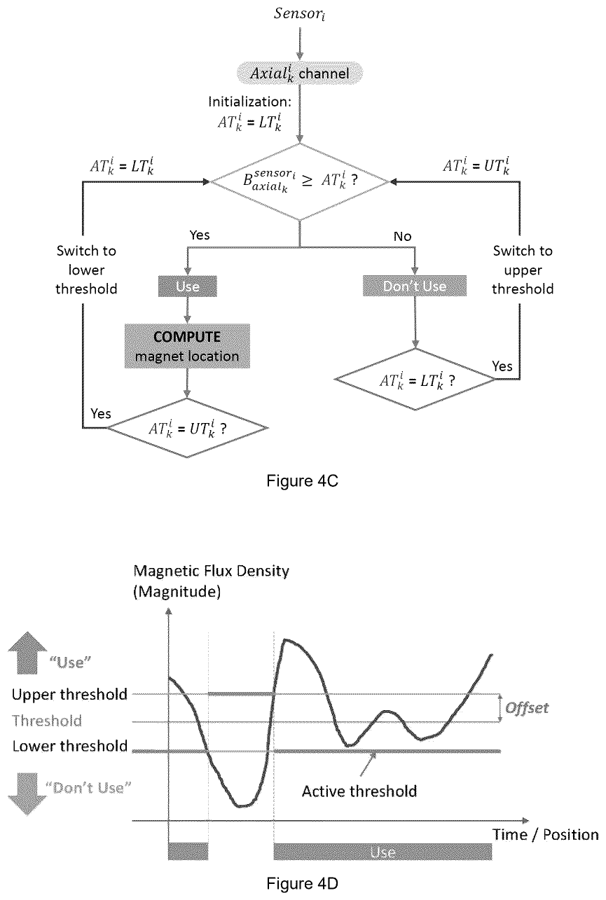

FIG. 4A shows a flowchart for the single-threshold scheme.

$${B}_{a x i a {l}_{k}}^{s e n s o {r}_{i}}$$

refers to the magnetic flux density read by the k^{th} axial magnetic field component or sensing channel of the i^{th} magnetic sensor 110. ATₖⁱ the magnetic field threshold for the respective axial direction of the respective i^{th} magnetic sensor 110. The magnetic field threshold ATₖⁱ is modelled after the noise profile of each respective axial direction of each i^{th} magnetic sensor 110, as shown in FIG. 4B, to determine the attentiveness of the respective k^{th} axial magnetic field component. A current k^{th} axial magnetic field component from an i^{th} magnetic sensor 110 is selected if the current k^{th} axial magnetic field component is at least equal to the magnetic field threshold ATₖⁱ for the corresponding axial direction of the corresponding i^{th} magnetic sensor 110. The selected k^{th} axial magnetic field component will be included in the computation of the magnet location and excluded otherwise.

A numerical simulation showed that only 76% (22208) of axial magnetic field components were selected under the single-threshold scheme in comparison to using all the axial magnetic field components (29214) in existing approaches. However, some axial magnetic field components of the magnetic sensors 110 that are positioned further away from the magnet 210 were selected due to the fluctuation of the magnetic field caused by environment noises. To minimize the inclusion of these axial magnetic field components, the double-threshold scheme may be used to improve selection of useful magnetic flux densities for magnet localization.

FIG. 4C shows a flowchart for the double-threshold scheme to determine the attentiveness of the respective k^{th} axial magnetic field component from each i^{th} magnetic sensor 110 while excluding fluctuating magnetic flux. As shown in FIG. 4D, the double-threshold scheme uses dynamic switching between a lower threshold LTₖⁱ and an upper threshold UTₖⁱ for each respective axial direction of each respective i^{th} magnetic sensor 110. The lower threshold LTₖⁱ and upper threshold UTₖⁱ have equal threshold offsets from a mean threshold. ATₖⁱ the magnetic field threshold for the respective axial direction of the respective i^{th} magnetic sensor 110. The magnetic field threshold ATₖⁱ is changeable between one of the lower threshold LTₖⁱ and the upper threshold UTₖⁱ depending on the current k^{th} axial magnetic field component from an i^{th} magnetic sensor 110.

For each k^{th} axial magnetic field component or sensing channel, the lower threshold LTₖⁱ is set as the active magnetic field threshold ATₖⁱ, i.e. ATₖⁱ=LTₖⁱ. This is to encourage the inclusion of potential low magnetic flux density read by the k^{th} axial magnetic field component before the magnet 210 approaches the respective i^{th} magnetic sensor 110. Once the current magnetic flux density drops below the lower threshold LTₖⁱ, the upper threshold will become active and set as the active magnetic field threshold ATₖⁱ, i.e. ATₖⁱ=UTₖⁱ. This is to filter out the fluctuation of magnetic flux density affected by environment noises. Hence, the magnetic field threshold ATₖⁱ is changeable from the lower threshold LTₖⁱ to the upper threshold UTₖⁱ if the current k^{th} axial magnetic field component is lower than the lower threshold LTₖⁱ. If the active magnetic field threshold ATₖⁱ is equal to the upper threshold UTₖⁱ and the current magnetic flux density rises to or above it, then the active magnetic field threshold ATₖⁱ switches to the lower threshold LTₖⁱ, i.e. ATₖⁱ=LTₖⁱ. Hence, the magnetic field threshold ATₖⁱ is changeable from the upper threshold UTₖⁱ to the lower threshold LTₖⁱ if the current k^{th} axial magnetic field component is at least equal to the upper threshold UTₖⁱ.

After selecting the axial magnetic field components, the processor calculates a pose of the magnet 210 using the selected axial magnetic field components and predetermined magnetic moments for the magnetic sensors 110. Notably, the pose describes a position and an orientation of the magnet 210 in six degrees of freedom in three-dimensional space. The processor tracks the device 200 using the calculated poses of the magnet 210 as the device 200 moves along the trajectory 220.

A numerical simulation was done to evaluate the apparatus 100 and in particular the threshold schemes in selecting useful axial magnetic field components for magnet localization. Theoretical magnetic field measurements were computed using Equation 1, and the total magnetic dipole model for each magnetic sensor 110 was obtained from the sensor calibration process. The optimal transverse offset of 1 mm and longitudinal offset of 0 mm were used in evaluating the localization performance using the single-threshold and double-threshold schemes. Random noise based on the actual noise characteristics is added to the simulation data.

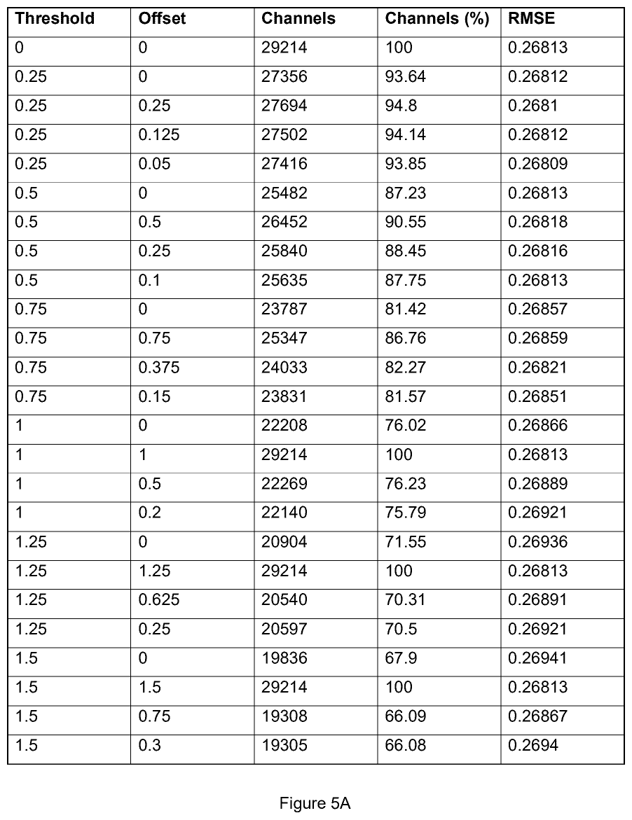

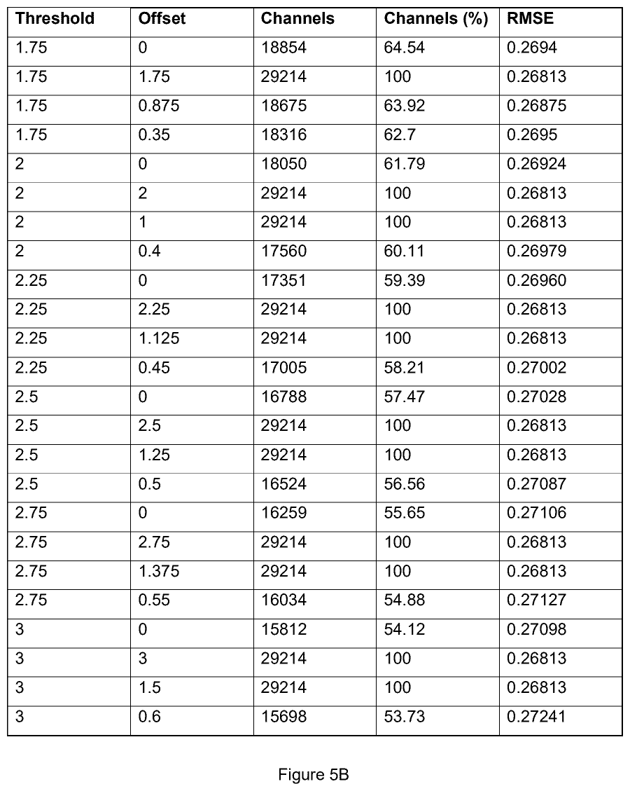

FIGS. 5A and 5B show the comparison of sensing resource utilization and RMSE between utilizing all axial magnetic field components or sensing channels and selecting only attentive sensing channels through the single-threshold or double-threshold schemes. The magnetic field threshold ranges from 0 to 3 which is equivalent to three standard deviations of noise. The threshold offset indicates which threshold scheme is active. The single-threshold scheme has an offset value of 0 whereas the double-threshold scheme has a positive offset value. The lower the RMSE, the higher the localization accuracy.

With both threshold schemes, it was found that the number of axial sensing channels utilized for localization reduces with the increase of threshold values. However, having lesser data of magnetic flux density does not affect the localization accuracy (p-value<0.5) as those being filtered out were measured by magnetic sensors 110 positioned further away from the magnet 210. The same localization accuracy (RMSE=0.26813) can be achieved using 87.23% of the axial sensing channels under the single-threshold scheme (threshold=0.5) or 87.75% of the axial sensing channels under the double-threshold scheme (threshold=0.5, offset=0.1) in comparison to the existing approach of using all axial sensing channels (threshold=0, offset=0). Notably, the localization accuracy (RMSE=0.26809) using the double-threshold scheme (threshold=0.25, offset=0.05) is higher than the existing approach but only 93.85% of the axial sensing channels were utilized. The numerical simulation results show that the apparatus 100 can be used for real-time long-range tracking of the magnet 210 (or the device 200 having the magnet 210) using lesser sensing resources while maintaining high localization accuracy.

By filtering only useful magnetic flux density using attentive axial sensing channels and the threshold schemes, less computational resource is spent on localizing the magnet 210. Indeed, only 53.73% (15698 of 29214) of axial sensing channels were engaged in the localization when the double-threshold scheme (threshold=3 SD, offset=0.6) was active. However, using lesser magnetic flux densities may come at a cost of poorer localization accuracy. This trade-off between saving computational resources and poorer localization accuracy provides flexibility for real-world applications with different requirements and constraints. For example, applications that require high accuracy can choose a threshold scheme with a lower RMSE, such as the double-threshold scheme (threshold=0.25, offset=0.05) with 93.85% axial sensing resource utilization and an RMSE of 0.26809. For example, applications with less computational resources can choose the single-threshold scheme (threshold=1) with 76.02% axial sensing resource utilization and an RMSE of 0.26866.

More magnetic sensors 110 can be added to increase the range of tracking the magnet 210 and to improve the robustness of the apparatus 100. If a magnetic sensor 110 is faulty, the apparatus 100 can still focus on utilizing the axial magnetic field components from nearby magnetic sensors 110 while ignoring the faulty ones. This ensures the uptime of the apparatus 100 so that the magnet 210 can be continuously tracked. This is important especially if the device 200 having the magnet 210 is a medical instrument that is inserted into the human body.

A real experiment was done using the apparatus 100 as shown in FIG. 1A. The apparatus 100 includes an array of 18 magnetic sensors 110, a permanent magnet 210, a Teensy microcontroller as the processor, and a gantry system 120 for controlling movement of the magnet 210. The magnet 210 is a composite of 6 cylindrical magnetic elements (R211-N52 by K&J Magnetics, Inc.) connected in series and aligned to the horizontal longitudinal direction. The magnet 210 has a diameter of 3.18 mm and a total length of 9.54 mm, and is sized according to the inner diameter of most nasogastric tubes for adults (size 14FR), which is one of the embodiments of the device 200 being a medical instrument.

The 18 magnetic sensors 110 are the MLX90393 3-axis magnetometers by Adafruit and are selected because of its wide range (±5 mT to ±50 mT) in all 3 axes) in measuring the magnetic field while being at an economic cost. The magnetic sensors 110 are supported on a 3D-printed support structure 112 and are arranged in an alternate/zig-zag pattern, spanning a distance of 430 mm. The transverse offset between the centre of each magnetic sensor 110 and the longitudinal direction is 6.5 mm and this was selected based on the sensor length (26 mm). The longitudinal offset between adjacent magnetic sensors 110 is 7.3 mm and this was selected such that the sensor array covers the distance of 430 mm, which is approximately the distance from below the neck to the belly button of the anatomical human model 300 as shown in FIG. 1B.

The apparatus 100 also includes four multiplexers 130 (TCA9548A by Adafruit) that were used to connect and interface all the magnetic sensors 110 with the processor through the 12C serial communication protocol. The gantry system 120 is configured to move the magnet 210 along the longitudinal x-axis and transverse y-axis. The magnet 210 is coupled to the gantry system 120 and is positioned about 40 mm above (along the z-axis) the sensor array and 150 mm away (along the x-axis) from the front of the sensor array. In this experiment, the gantry system 120 moved the magnet 210 horizontally along the longitudinal direction across the magnetic sensors 110.

The magnet 210 is controlled to move along a straight trajectory 220 from −150 mm to 580 mm to cover the distance of the sensor array (430 mm). For each i^{th} magnetic sensor 110, three axial magnetic flux densities (Bₓⁱ, B_{y}ⁱ, and B_{z}ⁱ) were measured by the i^{th} magnetic sensor 110. For N number of magnetic sensors 110, 3×N number of axial magnetic flux densities can be obtained to calculate the pose of the magnet 210 by solving the non-linear Equation 3 above.

The process of moving the magnet 210, measuring the magnetic field by the magnetic sensors 110, and independently recording the poses of the magnet 210 for ground truth comparison was automated using a customized Arduino program. The program enables the processor to send control signals to the gantry system 120 for moving the magnet 210 and receive magnetic field measurements from the magnetic sensors 110 via the multiplexers 130. The magnetic field measurements and the independent information of the magnet poses were recorded by the program for every 0.5 mm movement of the magnet 210.

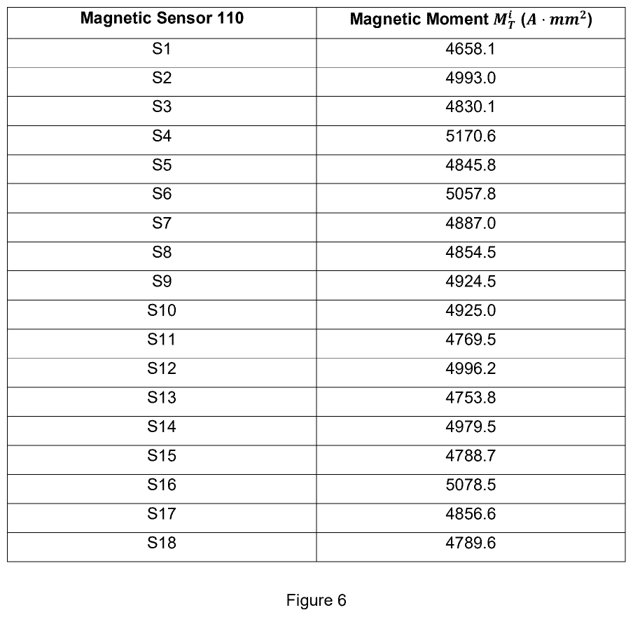

Further, the magnetic sensors 110 were calibrated beforehand to determine and optimize the magnetic moment of the magnet 210 on each magnetic sensor 110, since the magnetic moments were used to compute the poses of the magnet 210. The actual poses of the magnet 210 and the magnetic field measurements by the magnetic sensors 110 were fed into Equation 1 to solve for the magnetic moments, which were optimized by optimized by iteratively adjusting the magnetic moments to minimize the RMSEs. FIG. 6 shows the optimized magnetic moment for each of the 18 magnetic sensors 110.

The magnetic field measured by the magnetic sensors 110 was validated using a Gauss Meter, which independently measured the magnetic field around the magnet 210 at a specific location. Before the Gauss Meter makes a measurement to validate for a magnetic sensor 110, the magnetic sensor 110 was removed and replaced by a sensing probe of the Gauss Meter. The two magnetic sensors 110 adjacent to the removed one were also removed to allow sufficient space for the sensing probe. The Gauss Meter to measure the magnetic field and the gantry system 130 to move the magnet 210 were simultaneously activated. As with the magnetic field measurements from the magnetic sensors 110, the Gauss Meter measured the magnetic field for every 0.5 mm movement of the magnet 210.

Further, noise data of the magnetic sensors 220 were collected at specific positions along the entire range of the magnet trajectory 220. At each position, 500 measurements of the magnetic flux density from all the magnetic sensors 110 were performed. The standard deviation (SD) of each k^{th} axial magnetic field component from each i^{th} magnetic sensor 110 was computed using Equation 5.

$${σ}^{k} = \sqrt{\frac{1}{N} {( {b}_{i}^{k} - {μ}^{k} )}^{2}} \qquad ( Equation 5 )$$

σ^{k} represents the environment noise and other interference causing the k^{th} axial magnetic field component to fluctuate from the mean. N is the total number of samples. bᵢ^{k} is the magnetic flux density of the k^{th} axial magnetic field component from the i^{th} magnetic sensor 110. μ^{k} represents the mean magnetic flux density of the k^{th} axial magnetic field component. Notably, k can be the x-axis, y-axis, or z-axis of the magnetic field.

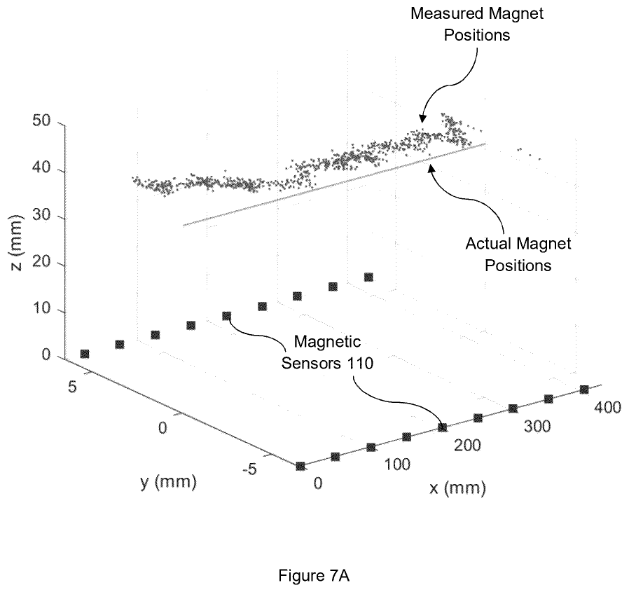

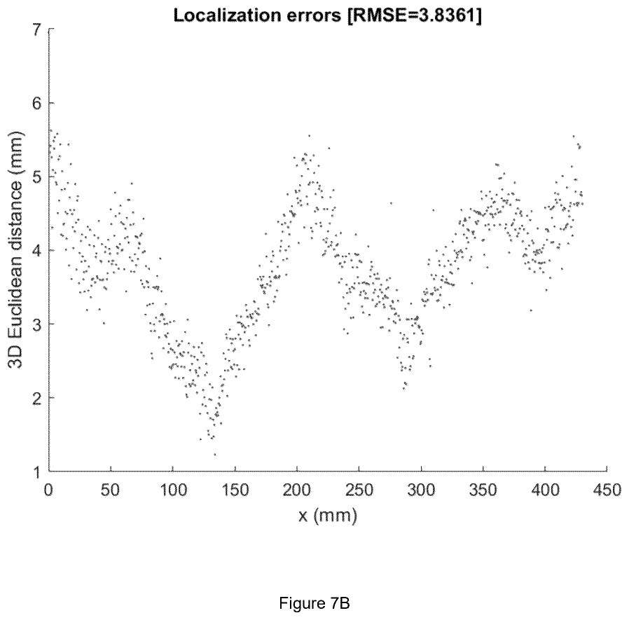

FIGS. 7A and 7B show the localization results and errors from the real experiment when the magnet 210 moved over a distance of 430 mm. The highest localization error is 6.95 mm (RMSE=3.8361 mm) and the average localization error (RMSE=3.8361) is comparable to existing magnet-based localization methods. However, the apparatus 100 is able to perform long-range tracking of the magnet 210 and any device 200 having the magnet 210, and the tracking range can go beyond the 430 mm that was experimented. The long-range tracking can be done without constraining the position of the magnet 210 within a defined area nor a minimal distance from the magnetic sensors 110. Moreover, with the selection of attentive axial magnetic field components, the computational resources required for data processing and magnetic localization can be significantly reduced.

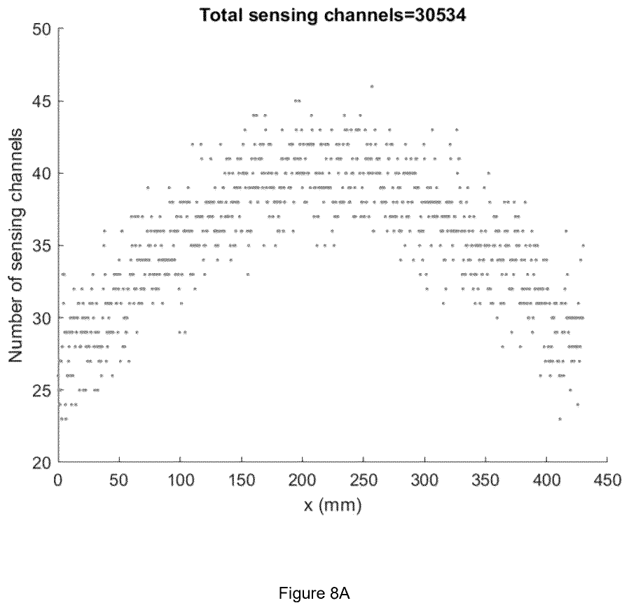

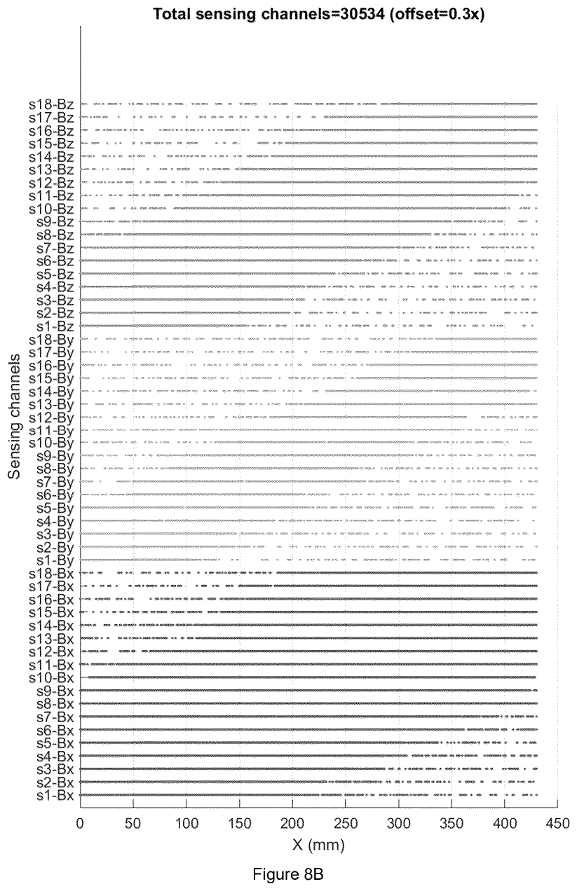

FIG. 8A shows the total number of attentive axial sensing channels selected to estimate the location of the magnet 210 when it traversed along the trajectory 220 of 430 mm distance. FIG. 8B shows which attentive axial sensing channels and their respective magnetic sensors 110 being selected to localize each magnet location. The total number of attentive axial sensing channels is 30534 channels, which is about 66% of the total number of axial sensing channels. For each pose of the magnet 210, the pose can be estimated using a maximum of 3×18=54 axial sensing channels, but the experiment results show that the actual number of axial sensing channels used ranges from 23 to 46 with a mean of 35. Overall, the attentive sensing channels used to track the magnet 210 range from 50% to 91% of the total number of axial sensing channels.

Another experiment was done to evaluate the apparatus 100 with the anatomical human model 300 to track the device 200 inserted therein, as shown in FIG. 1B. The device 200 is a nasogastric tube having the magnet 210 embedded at the instrument tip. The apparatus 100 includes an array of the magnetic sensors 110 and LEDs 310 aligned with the array. The magnetic sensors 110 and LEDs 310 are placed on the chest of the anatomical human model 300. The LEDs 310 facilitate visualization of the location of the magnet 210 when the nasogastric tube is inserted. The first LED 310 is located near then neck and depicts the magnet 210 entering the cervical oesophagus area. Each LED 310 blinks when the magnet 210 is near the LED 310 to reflect the estimated magnet position, so that the user can gauge the estimated position of the nasogastric tube tip during both the insertion and removal procedures.

The apparatus 100 and method described in various embodiments herein are able to perform real-time long-range tracking of a device 200 having a magnet 210 with good localization accuracy, resource conservation, and high robustness. To reduce computational cost, attentive axial magnetic field components are selected from the magnetic field measurements from the magnetic sensors 110 using suitable threshold schemes. Results from the numerical simulations and real experiments show that fewer but more attentive axial magnetic field components can be used to track the magnet 210 with lesser computational resources while achieving high localization accuracy.

The tracking range of the apparatus 100 can be adjusted by adding or removing magnetic sensors 110. Although more magnetic sensors 110 would result in more magnetic field data to be processed, the threshold schemes to select only the useful attentive axial magnetic field components reduce computational costs and improve computational efficiency. The long-range tracking of the apparatus 100 addresses the constraints of existing low-cost magnetic sensors which have a maximum tracking range of 250 mm.

The apparatus 100 can be used in various applications, including in the medical industry where real-time position confirmation of a medical instrument moving through the human body is desirable in medical diagnosis and treatment procedures. In particular, the apparatus 100 can be used to track the tip of medical instruments inside the human body using a permanent magnet 210, magnetic sensors 110, and a visual display, such as the LEDs 310 with similar length as the required tracking trajectory 220. The apparatus 100 may be paired with smart mobile or wearable devices to track the medical instruments. Possible applications include smart feeding tubes which would provide intuitive and real-time visual aid while a plastic tube is inserted through the nose down into the stomach/lungs prior to nutrition feeding or drug administration. With smart sensing and visual confirmation of the tube tip position, incidences of misalignment, complications, or radiation exposure, which are often caused by existing tube tip position verification methods such as pH paper tests, biochemical markers, or X-rays, can be reduced or avoided. In addition, critical delays and additional costs caused by the need for specialized equipment and trained personnel can be mitigated.

In the foregoing detailed description, embodiments of the present disclosure in relation to an apparatus and method for tracking a device having a magnet are described with reference to the provided figures. The description of the various embodiments herein is not intended to call out or be limited only to specific or particular representations of the present disclosure, but merely to illustrate non-limiting examples of the present disclosure. The present disclosure serves to address at least one of the mentioned problems and issues associated with the prior art. Although only some embodiments of the present disclosure are disclosed herein, it will be apparent to a person having ordinary skill in the art in view of this disclosure that a variety of changes and/or modifications can be made to the disclosed embodiments without departing from the scope of the present disclosure. Therefore, the scope of the disclosure as well as the scope of the following claims is not limited to embodiments described herein.

1. An apparatus for tracking a device having a magnet, the apparatus comprising:
a plurality of magnetic sensors arranged along a longitudinal direction based on a trajectory of the device, the magnetic sensors configured for measuring a magnetic field from the magnet as the device moves along the trajectory; and
a processor configured for:
receiving, from the magnetic sensors, the magnetic field measurements of the magnet;
determining, for each magnetic sensor, axial magnetic field components from the magnetic field measurements from the respective magnetic sensor, the axial magnetic field components directed along mutually orthogonal axial directions of the magnetic sensor;
selecting, for each magnetic sensor, the axial magnetic field components that satisfy a magnetic field threshold predefined for each axial direction of each magnetic sensor;
calculating a pose of the magnet using the selected axial magnetic field components and predetermined magnetic moments for the magnetic sensors; and
tracking the device using the calculated poses of the magnet as the device moves along the trajectory.

2. The apparatus according to claim 1, wherein the processor is further configured for calibrating the magnetic sensors with the magnet to determine the magnetic moment for each magnetic sensor.

3. The apparatus according to claim 2, wherein the processor is further configured for calibrating the magnetic sensors with the magnet to optimize the magnetic moment for each magnetic sensor by iteratively adjusting the magnetic moment to minimize root-mean-square error respect to a known trajectory of the magnet.

4. The apparatus according to claim 1, wherein the processor is further configured for calibrating the magnetic sensors with the magnet by adjusting the arrangement of the magnetic sensors.

5. The apparatus according to claim 4, wherein adjusting the arrangement of the magnetic sensors comprises adjusting transverse offsets of the magnetic sensors from the longitudinal direction and/or adjusting longitudinal offsets of the magnetic sensors along the longitudinal direction.

6. The apparatus according to claim 1, wherein the magnetic sensors are alternately arranged along the longitudinal direction, each magnetic sensor having a transverse offset from the longitudinal direction.

7. The apparatus according to claim 6, wherein the transverse offsets of the magnetic sensors are in equal magnitudes along alternate transverse directions.

8. The apparatus according to claim 1, wherein adjacent magnetic sensors have longitudinal offsets from each other in equal magnitudes along the longitudinal direction.

9. The apparatus according to claim 1, wherein a current axial magnetic field component from a magnetic sensor is selected if the current axial magnetic field component is at least equal to the magnetic field threshold for the corresponding axial direction of the corresponding magnetic sensor.

10. The apparatus according to claim 9, wherein the magnetic field threshold is changeable between one of a lower threshold and an upper threshold depending on the current axial magnetic field component.

11. The apparatus according to claim 10, wherein:
the magnetic field threshold is changeable from the lower threshold to the upper threshold if the current axial magnetic field component is lower than the lower threshold; and
the magnetic field threshold is changeable from the upper threshold to the lower threshold if the current axial magnetic field component is at least equal to the upper threshold.

12. A method for tracking a device having a magnet, the method comprising:
arranging a plurality of magnetic sensors along a longitudinal direction based on a trajectory of the device;
controlling movement of the device along the trajectory;
measuring, by the magnetic sensors, a magnetic field from the magnet as the device moves along the trajectory;
receiving, by a processor and from the magnetic sensors, the magnetic field measurements of the magnet;
determining, by the processor and for each magnetic sensor, axial magnetic field components from the magnetic field measurements from the respective magnetic sensor, the axial magnetic field components directed along mutually orthogonal axial directions of the magnetic sensor;
selecting, by the processor and for each magnetic sensor, the axial magnetic field components that satisfy a magnetic field threshold predefined for each axial direction of each magnetic sensor;
calculating, by the processor, a pose of the magnet using the selected axial magnetic field components and predetermined magnetic moments for the magnetic sensors; and
tracking, by the processor, the device using the calculated poses of the magnet as the device moves along the trajectory.

13. The method according to claim 12, further comprising calibrating, by the processor, the magnetic sensors with the magnet to determine the magnetic moment for each magnetic sensor.

14. The method according to claim 13, further comprising calibrating, by the processor, the magnetic sensors with the magnet to optimize the magnetic moment for each magnetic sensor by iteratively adjusting the magnetic moment to minimize root-mean-square error respect to a known trajectory of the magnet.

15. The method according to claim 12, further comprising calibrating, by the processor, the magnetic sensors with the magnet by adjusting the arrangement of the magnetic sensors.

16. The method according to claim 15, wherein adjusting the arrangement of the magnetic sensors comprises adjusting transverse offsets of the magnetic sensors from the longitudinal direction and/or adjusting longitudinal offsets of the magnetic sensors along the longitudinal direction.

17. The method according to claim 12, further comprising selecting, by the processor, a current axial magnetic field component from a magnetic sensor if the current axial magnetic field component is at least equal to the magnetic field threshold for the corresponding axial direction of the corresponding magnetic sensor.

18. The method according to claim 17, further comprising changing the magnetic field threshold between one of a lower threshold and an upper threshold depending on the current axial magnetic field component.

19. The method according to claim 18, further comprising:
changing the magnetic field threshold from the lower threshold to the upper threshold if the current axial magnetic field component is lower than the lower threshold; and
changing the magnetic field threshold from the upper threshold to the lower threshold if the current axial magnetic field component is at least equal to the upper threshold.

20. A computerized method for tracking a device having a magnet, the computerized method comprising:
receiving magnetic field measurements of the magnet as the device moves along the trajectory, the magnetic field measurements measured by a plurality of magnetic sensors arranged along a longitudinal direction based on the trajectory;
determining, for each magnetic sensor, axial magnetic field components from the magnetic field measurements from the respective magnetic sensor, the axial magnetic field components directed along mutually orthogonal axial directions of the magnetic sensor;
selecting, for each magnetic sensor, the axial magnetic field components that satisfy a magnetic field threshold predefined for each axial direction of each magnetic sensor;
calculating a pose of the magnet using the selected axial magnetic field components and predetermined magnetic moments for the magnetic sensors; and
tracking the device using the calculated poses of the magnet as the device moves along the trajectory.
